(19) United States
(12) Patent Application Publication
(10) Pub. No.: US 2026/0090760 A1
(43) Pub. Date: Apr. 2, 2026

(54) EXAMINING VISIBLE SKIN CONDITIONS

(71) Applicant: HM TECHNOLOGY INTERNATIONAL LIMITED, Middlesex (GB)

(72) Inventor: Hrand Mami MAMIGONIANS, London (GB)

(21) Appl. No.: 19/338,141

(22) Filed: Sep. 24, 2025

(30) Foreign Application Priority Data
Sep. 30, 2024 (GB) 2414309.1

(51) Int. Cl. A61B 5/00; A61B 5/107

(52) U.S. Cl. CPC A61B 5/441; A61B 5/0064; A61B 5/0077; A61B 5/1075; A61B 5/1079; A61B 5/742; A61B 2560/04; A61B 2576/02

(57) ABSTRACT

A method for examining visible skin conditions is shown. A housing is located at a region of a skin condition and image data of the skin over this region is captured. Electric fields are generated that penetrate the skin over the region, such that an application of the apparatus produces image-related signals and electric field related signals for the same skin condition. Depth of skin penetration of the skin condition may be assessed by calculating surface area of the skin condition from the image data and determining volume of the skin condition from the electric field related signals and processing the calculated surface area in combination with the determined indication of volume to assess the depth of skin penetration. The electric field generating device may comprise a first dielectric substrate having a hole and the capturing step may be performed by a camera viewing the skin condition through this hole.

### CROSS REFERENCE TO RELATED APPLICATIONS

This application claims priority from United Kingdom Patent Application No. 2414309.1, filed Sep. 30, 2024, the whole contents of which is incorporated herein by reference in its entirety.

### TECHNICAL FIELD

The present invention relates to an examination apparatus and in particular to an apparatus for examining visible skin conditions. The present invention also relates to an examination method and in particular to a method of examining visible skin conditions.

### BACKGROUND OF THE INVENTION

It is known to examine skin conditions visually, which may be achieved directly, possibly via a lens or indirectly by means of a camera. Suggestions have been made to the effect that a video camera included within a mobile phone could be deployed to achieve this. However, problems arise in terms of reliability and repeatability.

Proposals have been made for examining visible skin conditions using electric fields, However, problems arise if electrodes generating these electric fields are in contact with a skin condition itself.

### BRIEF SUMMARY OF THE INVENTION

In summary, there is provided a method for examining visible skin conditions, comprising the steps of: locating a housing at a region of a visible skin condition; capturing image data of the skin over said region; generating electric fields that penetrate the skin over said region, such that an application of the apparatus produces both image-related signals and electric field related signals for the same skin condition; assessing depth of skin penetration of the visible skin condition; and generating an output indicative of said assessing step. The method further performs the steps of the steps of: calculating the surface area of the visible skin condition from the image data produced by said capturing step; determining an indication of the volume of the visible skin condition from the electric field related signals; and processing said calculated surface area in combination with said determined indication of volume to assess the depth of skin penetration.

The electric field related signals provide data indicative of electrical properties of the visible skin condition. In particular, these electrical properties may comprise electrical conductivity, permittivity or a combination of these properties.

In an embodiment, the generating step is performed by an electric field generating device that comprises a first dielectric substrate having a hole having a first radius; the capturing step is performed by a camera; and the visible skin condition is viewed through this hole. The image data may be captured by the camera substantially at the same time as electric fields are generated by the electric field generating device.

In an embodiment, the calculating step is performed by pixel counting. The image data may be enhanced in response to the electric field data. For example, the image data may be changed by an extent related to the assessed depth of penetration.

There is also provided an apparatus for examining visible skin conditions, comprising a housing locatable at a region of a skin condition. The housing contains a camera for capturing image data of the skin over said region and an electric field generating device configured to generate electric fields that penetrate the skin over said region, such that an application of the apparatus produces both image related signals and electric field related signals for the same skin condition. The electric field generating device comprises a first dielectric substrate with a hole having a first radius; and the camera views the skin condition through said hole.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows an apparatus for examining visible skin conditions;

FIG. 2 shows the apparatus of FIG. 1 obtaining test data;

FIG. 3 shows the apparatus of FIG. 1 in an inverted position;

FIG. 4 shows the apparatus of FIG. 1 in a cradle;

FIG. 5 details the dielectric substrate identified in FIG. 3;

FIG. 6 shows an internal subassembly for the apparatus identified in FIG. 1;

FIG. 7 shows procedures performed by a microcontroller contained within the apparatus;

FIG. 8 details procedures for producing electric field data as identified in FIG. 7;

FIG. 9 shows procedures for calculating and saving data identified in FIG. 8;

FIG. 10 illustrates the production of output samples identified in FIG. 9;

FIG. 11 details procedures for identifying and storing information identified in FIG. 9;

FIG. 12 illustrates the population of a database table;

FIG. 13 shows the examining apparatus connected to a data processing system;

FIG. 14 shows procedures performed by the data processing system identified in FIG. 13;

FIG. 15 details procedures for processing the received data identified in FIG. 14;

FIG. 16 illustrates the processing of images;

FIG. 17 shows an image of a first visible skin condition;

FIG. 18 shows an image of a second visible skin condition

FIG. 19 shows a procedure for processing image and electric field related data; and

FIG. 20 shows a cross section of the apparatus of FIG. 3.

### DETAILED DESCRIPTION OF EMBODIMENTS OF THE INVENTION

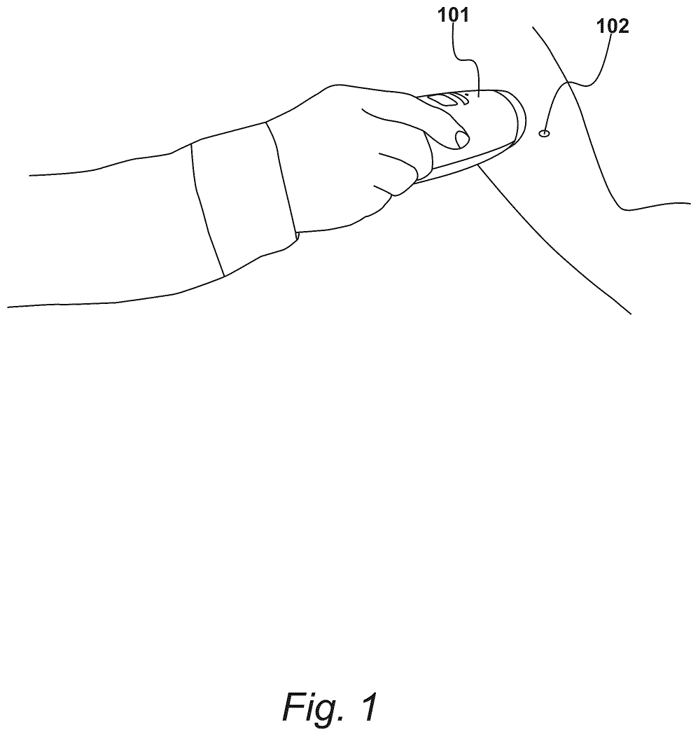

### FIG. 1

An apparatus for examining visible skin conditions is shown in FIG. 1 and comprises a housing 101 locatable at a region of the skin under examination; an example 102 of a skin condition is illustrated. The housing contains a camera for capturing image data of the skin over the region of the skin condition. In addition, an electric field generating device is configured to generate electric fields that penetrate the skin over the region, such that an application of the apparatus produces both image related signals and electric field related signals for the same skin condition.

In an embodiment, reference image data is produced and reference electric field data is also produced by placing the apparatus firstly at a position of healthy skin, as shown in FIG. 1. Thus, while located as illustrated in FIG. 1, a camera captures reference image data and the electric field generating device produces reference signals from which electric field reference data is produced.

In an alternative embodiment, as an alternative to using a region of skin for producing reference data, reference data may be obtained by deploying the apparatus upon a reference proxy device having known optical and electrical properties. As an example, the reference proxy device may comprise a receptacle containing an oil, such as olive oil.

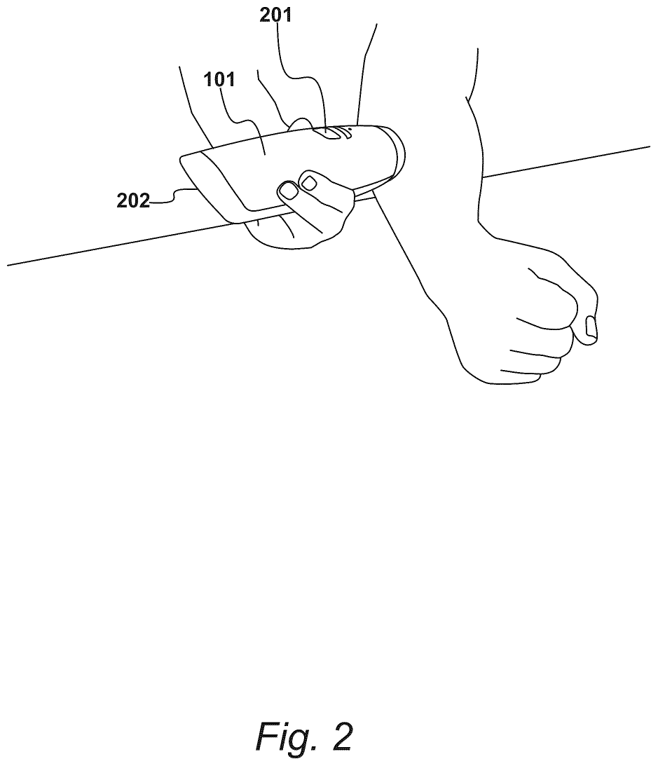

### FIG. 2

As shown in FIG. 2, after obtaining reference data, as described with respect to FIG. 1, the apparatus is moved to the position of the visible skin condition 102 to produce test image data and test electric field data. To initiate a procedure, an operative applies pressure to a manual activation button 201 to generate reference data. Thereafter, after moving the apparatus to a location substantially similar to that shown in FIG. 2, a second activation of this button 201 results in the generation of the test data. In alternative embodiments, alternative measures may be taken to initiate this activation and activation could be initiated by a connected computer.

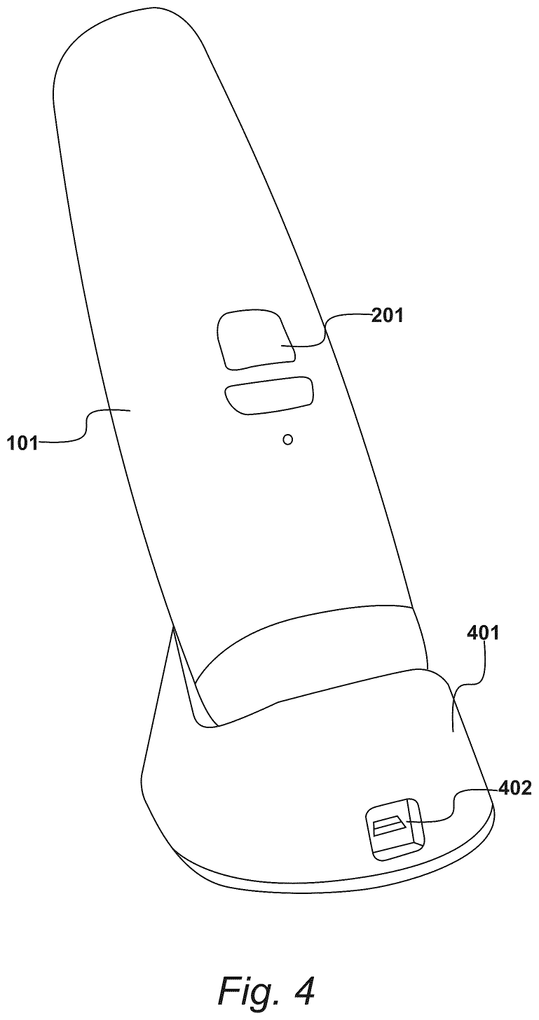

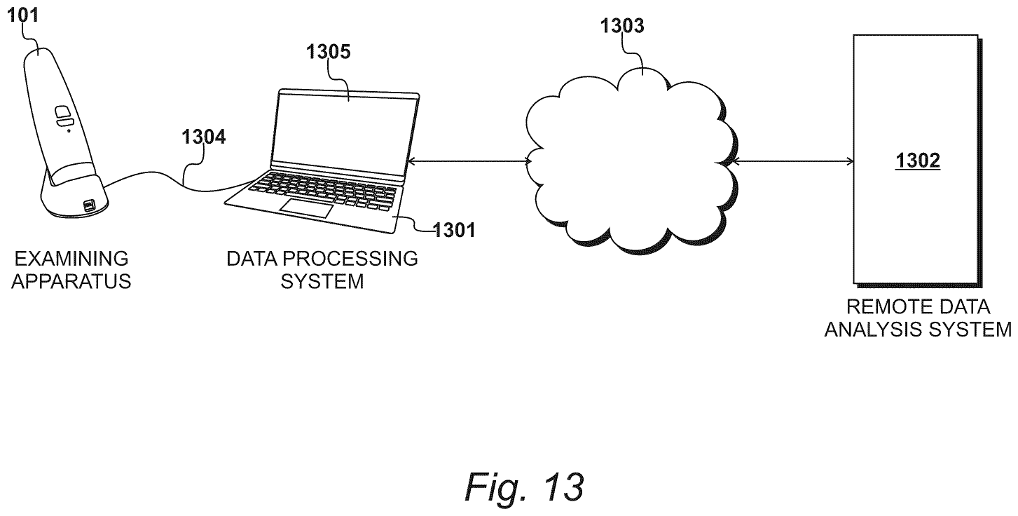

In the embodiment shown in FIG. 2, the apparatus includes a socket 202 for interfacing with a cradle, as described with reference to FIG. 4, which may in turn be connected to a data processing system, such as a laptop computer and a connection of this type will be described with reference to FIG. 13.

In the embodiment of FIG. 2, the camera is configured to capture image data substantially at the same time as the electric field generating device generates electric fields. In practice, these operations are likely to be performed sequentially but a complete cycle may be completed within a time interval of only a few seconds. Thus, from the perspective of an operative, the electric field data and the image data are captured substantially at the same time, such that the captured data is derived from the apparatus held in a fixed location relative to the region of the skin being examined or the region of the skin providing reference data.

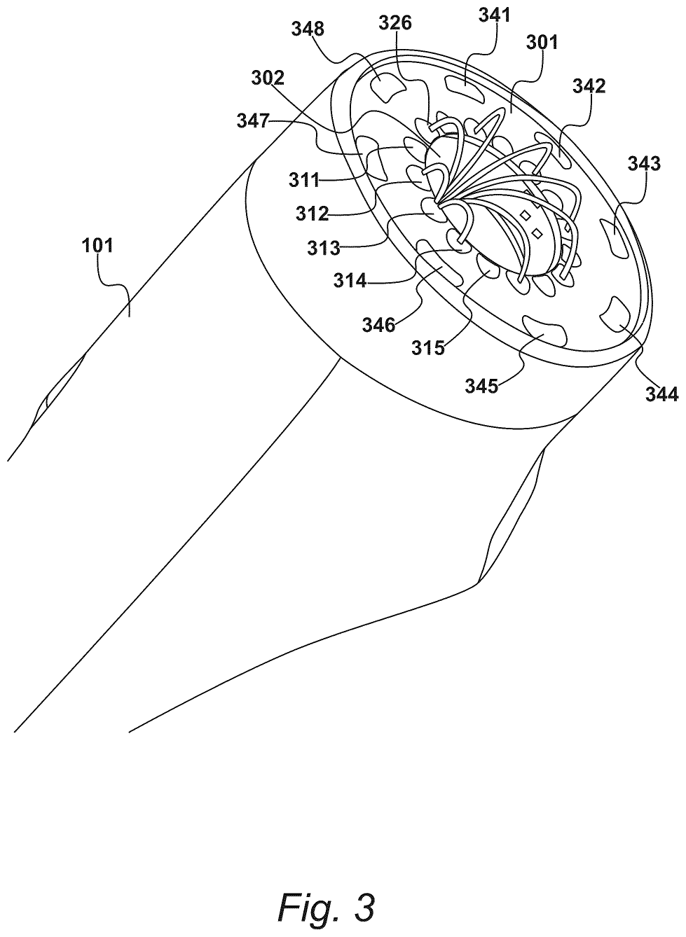

### FIG. 3

The housing 101 is shown inverted in FIG. 3, exposing a first dielectric substrate 301 with a hole 302 having a first radius. The dielectric substrate may take the form of a board of a substantially conventional type onto which circuits may be printed or etched. Scanning electrodes, including a first scanning electrode 311 and a second scanning electrode 312, are circumferentially evenly displaced on a first planar surface of the dielectric substrate 301 at a second radius around the hole 302. In this way, the camera views the skin condition through the hole 302. This portion of the housing, which comes into contact with a subject's skin, may be covered by a transparent replaceable cap that is also configured to permit the transmission of electric fields.

In the embodiment shown in FIG. 3, the apparatus has a total of sixteen scanning electrodes, although alternative embodiments could have fewer or more scanning electrodes. In an embodiment, each scanning electrode may be energized as a transmitter or monitored as a receiver. Thus, in an embodiment, a full scanning cycle may consist of energizing each of the available scanning electrodes which are sequentially selected. In an embodiment, upon selecting a scanning electrode as a transmitter, it may be energized a total of fifteen times with a different scanning electrode being selected as the receiver for each energization.

In the embodiment of FIG. 3, each scanning electrode is configured exclusively to be energized, and thus act as a transmitter, or to be configured as a receiver. In the embodiment of FIG. 3, the functionality of the electrodes is fixed; with eight of the scanning electrodes acting as transmitters alternating with the remaining eight electrodes acting as receivers. In the example shown in FIG. 3, transmitter electrode 313 is being energized sequentially eight times. Scanning electrode 312 acts as a receiver and scanning electrode 314 acts as a receiver on the next energization cycle. Thus, eight energizations of the same transmitter electrode are made sequentially with each of the available eight receiving electrodes. Energizations of this type produce electric fields that extend away from the plane of the dielectric substrate 301.

In the embodiment of FIG. 3, respective electrical conductors for each of the scanning electrodes 311 to 326 are configured to pass through the first dielectric substrate to a second planer surface.

The embodiment of FIG. 3 also includes circumferentially and substantially evenly displaced secondary electrodes 341 to 348 etc. In the embodiment of FIG. 3, a total of eight secondary electrodes are provided at a third radius that is larger than the second radius. In an alternative embodiment, the number of secondary electrodes present is the same as the number of scanning electrodes and each secondary electrode may be radially displaced from a respective scanning electrode. The secondary electrodes 341 to 348 do not perform scanning operations as such and the scanning operations are performed exclusively by the scanning electrodes. However, appropriate connections to the secondary electrodes can influence the resulting electric fields and as such can increase the size of the dataset.

### FIG. 4

After performing the operations described with respect to FIG. 1 and FIG. 2, the apparatus is returned to a cradle 401. In an embodiment, the socket 202 engages a plug extending from the cradle 401 and the cradle 401 may be connected to a data processing system by means of a USB (or similar) socket 402. A USB connection from socket 402 may be used to perform data transfer from the apparatus to a data processing system.

In an alternative embodiment, data transfer from the apparatus to the data processing system occurs wirelessly, thereby enhancing an operative's ability to move around a subject and position the apparatus appropriately.

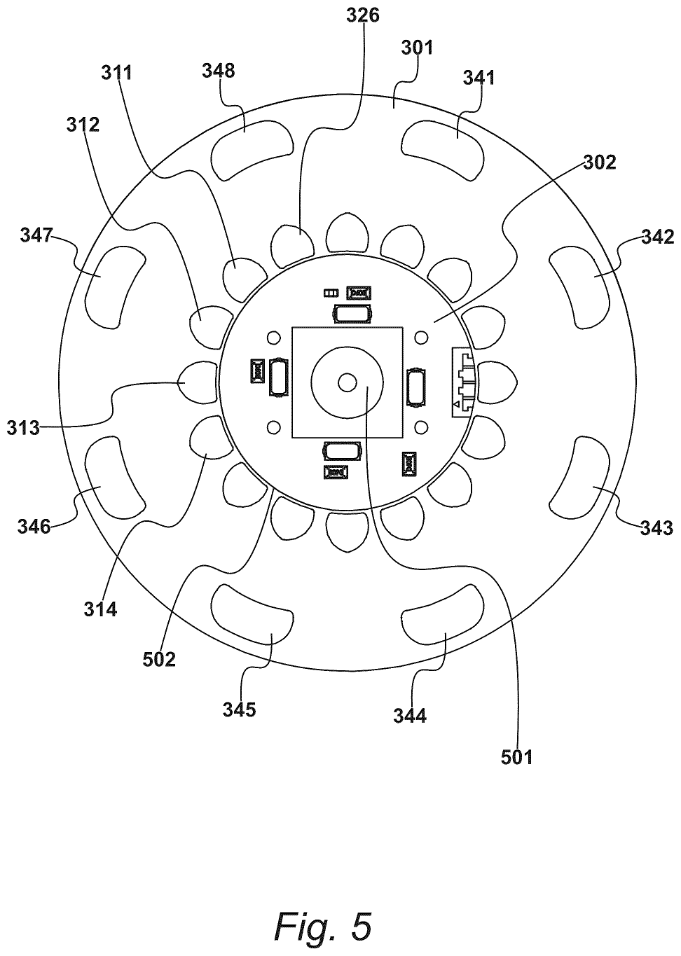

### FIG. 5

The dielectric substrate 301 is shown in FIG. 5, with the scanning electrodes 311 to 316 and the secondary electrodes 341 to 348 mounted thereon. A camera 501 is visible through the hole 302 and is surrounded by a cylindrical shroud 502.

The camera 501 may produce individual still images in response to being triggered. Alternatively, the camera may be a video camera producing a stream of video images which may be viewed on an appropriate external device while the apparatus is being placed in position. Activation of the manual activation button 201 then results in a particular image being selected followed by the electric fields being generated to produce electric field data.

In an alternative embodiment, it is also possible for video material to be recorded.

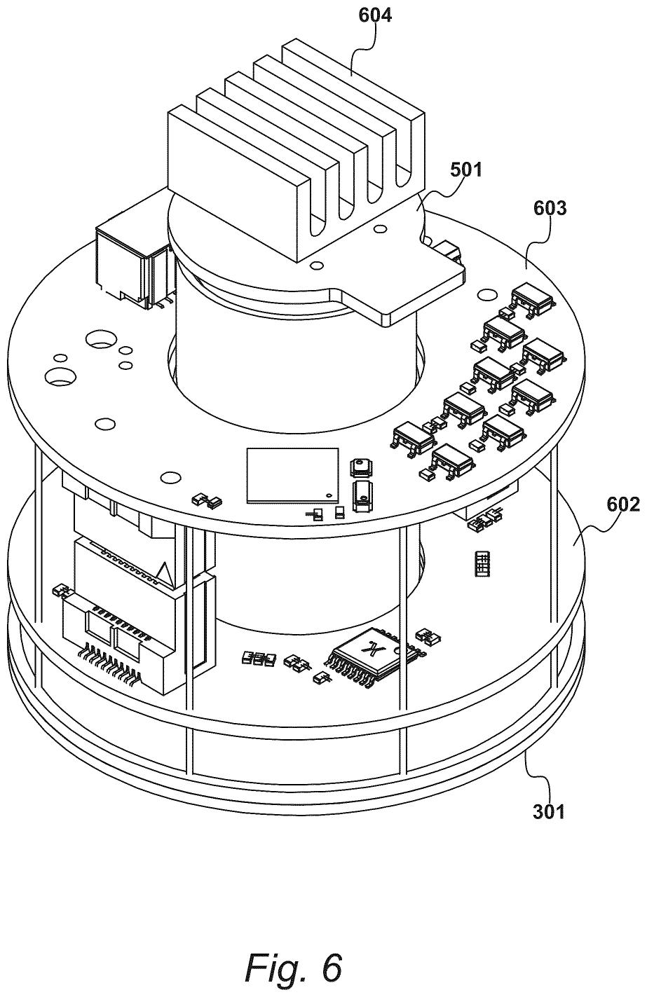

### FIG. 6

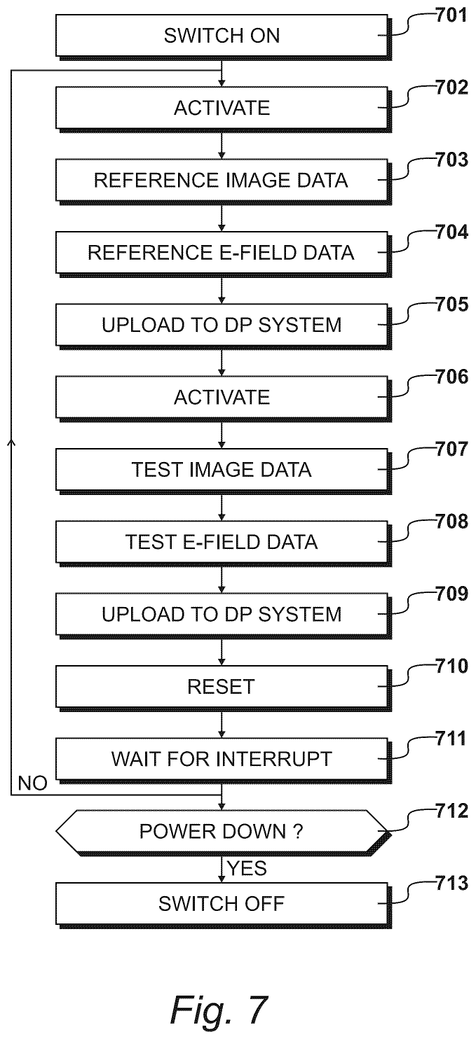

An internal subassembly of the apparatus is shown in FIG. 6, with the first dielectric substrate 301 inverted. The first dielectric substrate 301 may take the form of a conventional circuit board which, as shown in FIG. 6, is attached to a second circuit board 602 which is in turn attached to a third circuit board 603. Electronic components are supported on the second circuit board 602 and on the third circuit board 603 for implementing procedures described with reference to FIG. 7 to FIG. 12. In an embodiment, these electronic components may include an inertial module such as that produced by iNEMO and made available under the commercial designation LSM6 DS016IS. This provides a three-axis accelerometer and a three-axis gyroscope with an intelligent sensor processing unit. In this way, it is possible for a data processing system to receive data indicating the position and orientation of the apparatus when in use, as described with reference to FIG. 1 and FIG. 2.

The underside of camera 501 is also shown in FIG. 6, to which is attached a heatsink 604. In an embodiment, the camera is widely available under the commercial designation OV5640 and is capable of providing image data at a definition from 320×240 pixels up to 2592×1944 pixels in an eight-bit or ten-bit RAW RGB output format. An autofocusing capability is also available but the necessity for this is mitigated on the basis that the distance between the camera and the subject remains substantially the same; given that activation only takes place after the apparatus has been appropriately located.

### FIG. 7

Procedures performed by a microcontroller contained within the apparatus described with reference to FIG. 6, are detailed in FIG. 7. After being switched on at step 701, the apparatus is placed in a reference position, as described with reference to FIG. 1. The processor is then interrupted by activation of the manual activation button 201 at step 702. In an alternative embodiment, it is possible for a first thread to be responsible for generating image data and a second thread to be responsible for generating the electric field data. In the embodiment shown in FIG. 7, the reference image data is produced at step 703 and the reference electric field data is then produced at step 704. In an embodiment, a light-emitting diode present within the apparatus may be activated to indicate to an operative that the reference data has been collected. The operative would then be invited to relocate the apparatus to collect the test data, as described with reference to FIG. 2.

Meanwhile, in an embodiment, the reference image data and the reference electric field data are uploaded to a data processing system, as described with reference to FIG. 13. Thus, the light-emitting diode may be illuminated after the upload process has been completed and the procedure then anticipates a further activation of the manual activation button 201 at step 706.

At step 707, test image data is produced which is then followed, at step 708, by the production of test electric field data. These procedures are substantially similar to procedures 703 and 704 respectively. Thus, again, the test image data and the test electric field data are uploaded to the data processing system at step 709.

In the embodiment of FIG. 7, a reset operation is performed at step 710 in anticipation of a further interrupt signal being received. The processor therefore enters a wait state at step 711.

At step 712, a question is asked as to whether the system is to power down and if answered in the affirmative, the apparatus switches off at step 713 to conserve battery power. Alternatively, if the question asked at step 712 is answered in the negative, the processor awaits the next activation interrupt at step 702.

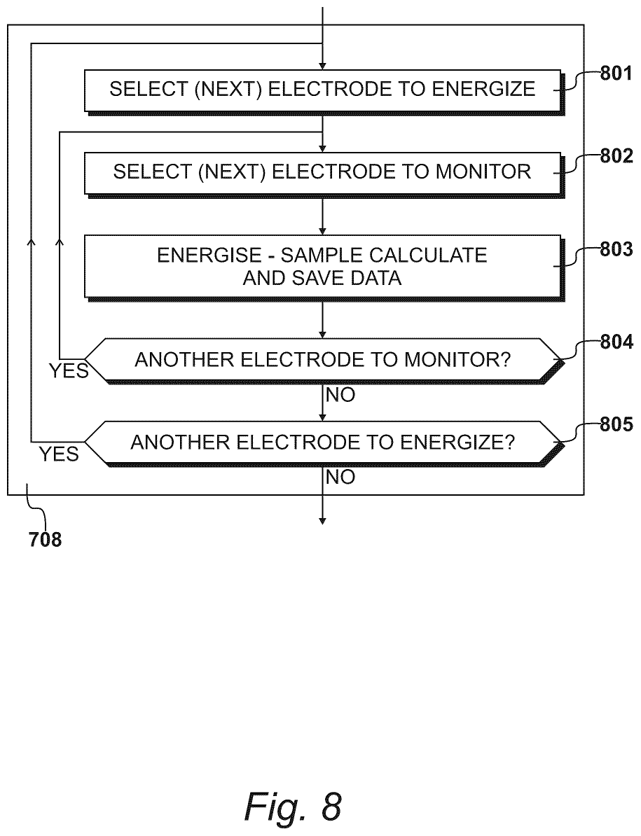

### FIG. 8

As described with reference to FIG. 7, procedure 704 for producing the electric field reference data and procedure 708 for producing the electric field test data are substantially similar; and procedure 708 is detailed in FIG. 8. In an embodiment, scanning electrodes may be selected for energization to optimize the quality of the collected data. Similarly, appropriate scanning electrodes may be monitored. In the embodiment illustrated in FIG. 8, all of the transmitter electrodes are selected as an energized electrode and for each selected energized electrode, all of the remaining receiver electrodes are sequentially selected as monitored electrodes. The selection of these capacitively coupled electrodes may occur in any order but to facilitate the creation of appropriate instructions, the electrodes are selected sequentially in numerical order in the embodiment shown in FIG. 8.

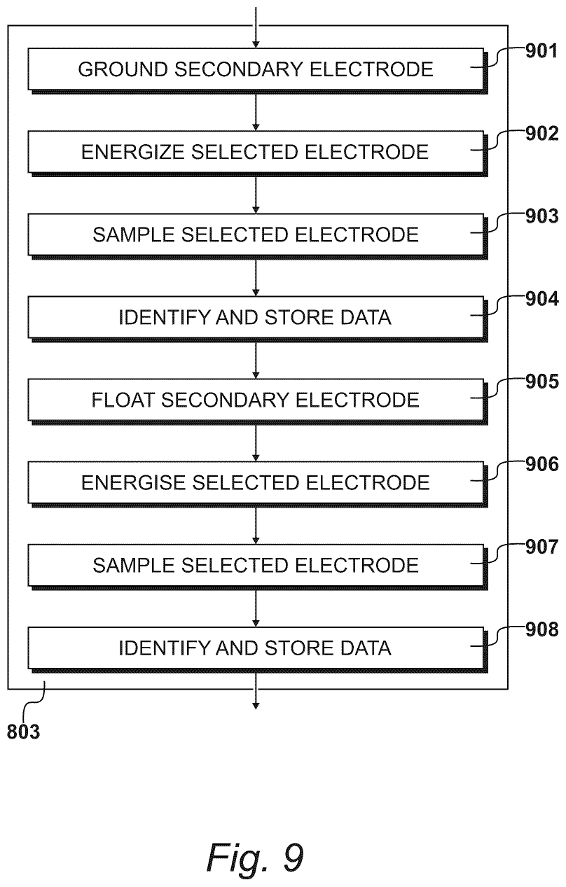

In the embodiment shown in FIG. 8, on a first iteration, the first transmitter electrode is selected at step 801 and the first receiver electrode is selected at step 802. At step 803, data is collected for this capacitively coupled pair as described with reference to FIG. 9.

At step 804, a question is asked as to whether another electrode is present to be monitored and on the first iteration this question will be answered in the affirmative resulting in the selection of the next receiver electrode. Thus, this procedure is repeated until all eight of the receiver electrodes have been selected, resulting in the question asked at step 804 being answered in the negative.

At step 805, a question is asked as to whether another transmitter electrode is present to be energized such that, on this first iteration, the next transmitter electrode will be selected at step 801 and repeated iterations of step 802 will result in all of the remaining receiver electrodes being sequentially selected as electrodes to be monitored. Thus, the process will continue until all of the transmitter electrodes have been selected at step 801 and the question asked at step 805 will be answered in the negative.

### FIG. 9

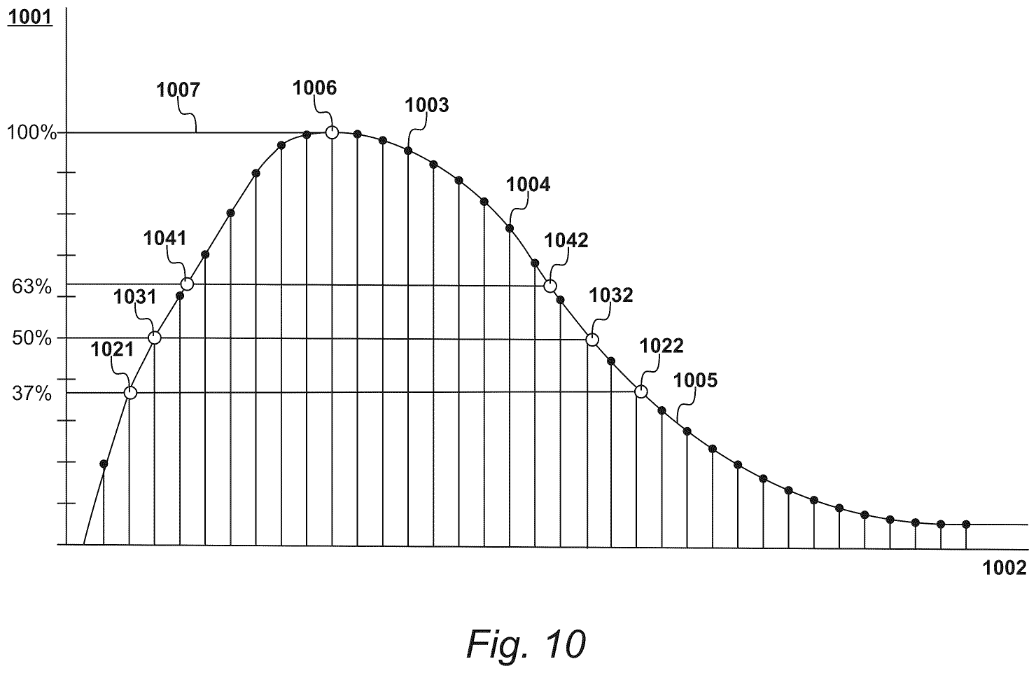

Procedures 803 for calculating and saving data derived from the electric field signals are detailed in FIG. 9. At step 901, one or more of the secondary electrodes 341 to 348 are grounded. At step 902 the scanning electrode selected at step 801 is energized and at step 903 the scanning electrode selected at step 802 is monitored and sampled. The data collected, as described with reference to FIG. 10, is then identified and stored at step 904.

At step 905, the secondary electrodes 341 to 348 are allowed to float and the selected electrodes are again coupled by energizing the electrode selected at step 801 and monitoring the electrode selected at step 802. Thus, each selected pair are capacitively coupled twice in accordance with this embodiment. Again, at step 908, the data is identified and stored.

In an embodiment, each energizing pulse lasts for a duration of ten microseconds and individual pulses are separated by a duration of ninety microseconds. During each cycle, one hundred samples are taken, thereby requiring a sample rate of five megahertz. In an alternative embodiment, fewer samples are taken and in alternative embodiments more samples may be taken. In an embodiment, fifty samples are taken.

An analog to digital converter, which in an embodiment forms part of the microcontroller, converts each sample into a twelve-bit representation and as a result of this, each energizing pulse generates a significant amount of data. However, the processor is fast enough to allow a significant amount of processing to take place during the sample period. Thus, by comparing samples, it is possible to identify a peak value and the regular intervals between samples allows the time at which this peak value occurred to be determined. Thus, each sample point is made up of data that defines a voltage level at a particular time.

The resulting data is saved initially at step 904 and then again at step 908. This raw data is processed to produce a smaller volume of output data that, in an embodiment, is transferred to a data processing system as described with reference to FIG. 13.

In an alternative embodiment, the raw data is transmitted to the data processing system and the procedures identified above, for reducing the data volume, are performed on the data processing system.

### FIG. 10

The production of output samples at step 903 (or at step 907) is illustrated in FIG. 10. In the graph shown in FIG. 10, output voltage 1001 is plotted against time 1002. The sampling operation creates data points, such as data point 1003 and data point 1004. As is known in the art, it is also possible to fit a curve 1005 to the real data points, such that other values on this curve may be calculated through a process of interpolation. From this, it is possible to identify a peak value 1006.

Having calculated the peak value 1006, it is then possible to identify points at which a portion of this peak value has been achieved. Thus, from the large volume of raw data, it is possible to calculate a much more limited volume of data (information) which conveys what is required in terms of a rate of charge, a peak and a rate of discharge. Furthermore, it is known that the absolute peak value and the rate of discharge are directly related to the conductivity and permittivity of the tissue being examined.

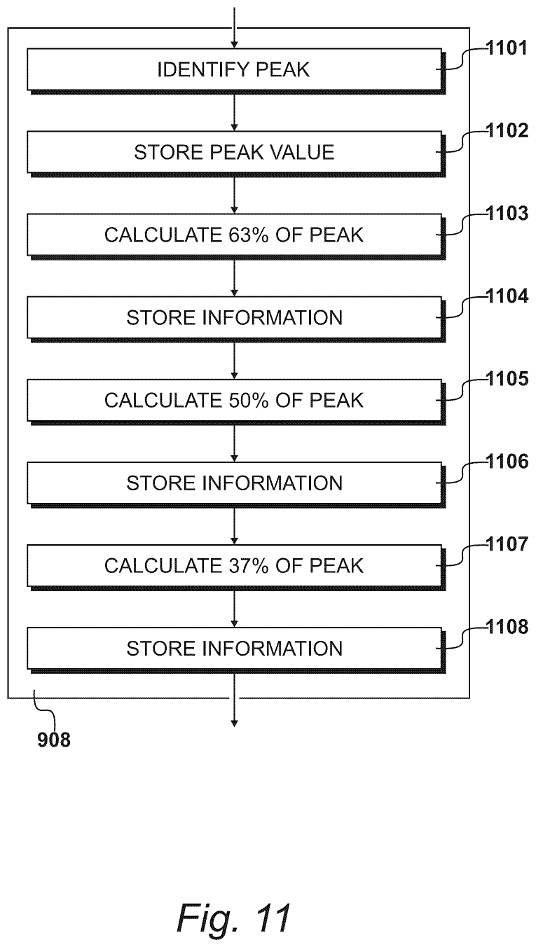

### FIG. 11

Procedures 904 and 908 for identifying and storing information (the data that will be uploaded to the data processing system) are shown in FIG. 11.

At step 1101, the peak value 1006 is identified and this peak information 1007 is stored at step 1102. At step 1103, a value is calculated that represents sixty-three percent of the peak value 1007. At step 1104, information is stored, consisting of a first data point 1021 and a second data point 1022 at which the curve 1005 passes through the sixty-three percent value. At step 1105, a value is calculated that represents fifty percent of the peak value 1007. A first data point 1031 and a second data point 1032 are identified where the curve 1005 crosses this fifty percent level. At step 1107, a value is calculated that represents thirty-seven percent of the peak value 1007. Again, at step 1108, a first data point 1041 is stored, along with a second data point 1042, showing where the curve 1005 crosses these values.

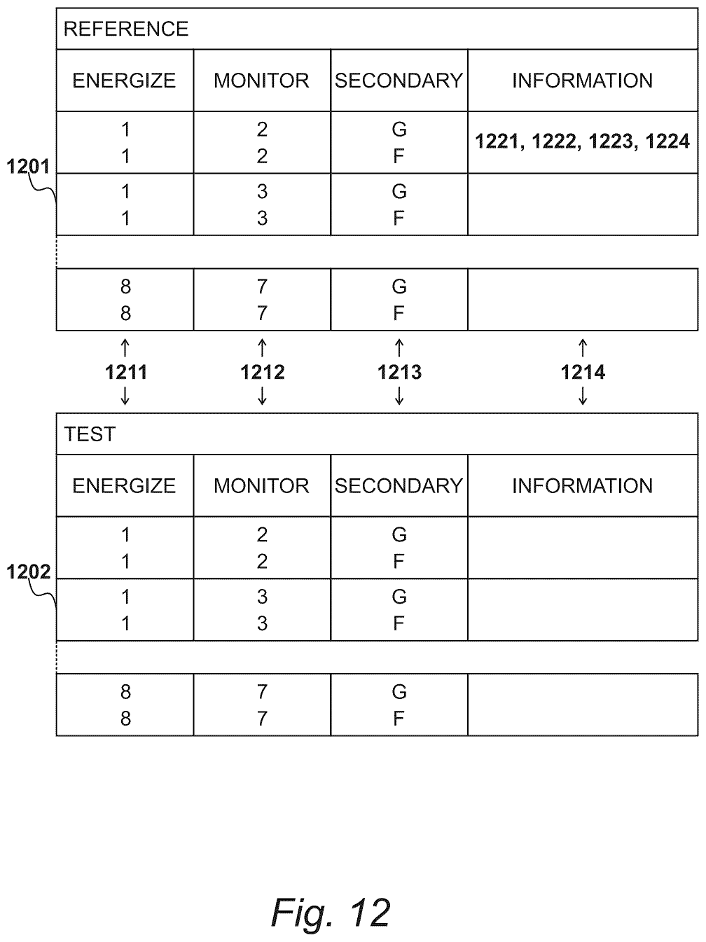

### FIG. 12

The refined data (information) calculated at steps 1102, 1104, 1106 and 1108 are stored in a database and a representation of this database is illustrated in FIG. 12. A data table 1201 is constructed for the reference data and a similar data table 1202 is constructed for the test data. In a first column 1211, the electrode being energized is recorded and in a second column 1212 the electrode being monitored is recorded. For each of these combinations, a third column 1213 records whether secondary electrodes were grounded or allowed to float. The resulting information is then stored in a fourth column 1214.

In this embodiment, the information (data for transmission to the data processing system) represents the first peak value 1221. The information then represents the two positions for thirty-seven percent of the peak value 1222, the two positions for fifty percent of the peak value 1223 and the two positions for sixty-three percent of the peak value 1224.

After fully populating the database table of FIG. 12, the resulting information consists of a relatively small volume compared to the totality of raw data generated through the scanning and sampling operations. In an embodiment, this information is transferred to a data processing system, as described with reference to FIG. 13, for subsequent processing. The overall objective is to identify the nature of the tissue under consideration. In particular, when scanning visible skin conditions, the overall objective is to give an indication as to whether the skin condition is considered benign or whether the skin condition is considered malignant and therefore requires further attention.

As is clear from the table shown in FIG. 12, similar combinations exist for both the reference stage 704 and the test stage 708. Thus, specific similar information entries may be directly compared to determine the extent to which they differ. In an embodiment, a larger difference may indicate that the skin under test has characteristics that differ significantly from healthy skin; thereby prompting further investigation.

### FIG. 13

The examining apparatus 101 is shown in FIG. 13 connected to a data processing system 1301. The data processing system 1301 also communicates with a remote data analysis system 1302 via the Internet 1303.

In the embodiment described with reference to FIG. 6, the apparatus includes a signal processor for processing electric field signals produced by the electric field generating device, which are in turn influenced by the electrical properties of the penetrated tissue, to produce electric field data as described with reference to FIG. 7 to FIG. 12. Thus, in an embodiment, the data stored in the database described with reference to FIG. 12 is transmitted to the data processing system 1301 either wirelessly or via a connected cable 1304. The cable 1304 may also be used to recharge batteries contained within the apparatus 101.

In an embodiment, data communication occurs wirelessly from the examining apparatus to the data processing system 1301 without the need for the examining apparatus to be returned to a cradle. In this way, it is possible for the examining apparatus 101 to be displaced by a significant distance away from the data processing system 1301; possibly being located in a different room, while allowing the data to be collected by the data processing system. In this alternative embodiment, the examining apparatus is returned to a cradle for charging purposes only and cable 1304 may be connected to a conventional power supply.

In addition to transferring the electric field data, image data is also transmitted from the apparatus 101 to the data processing system 1301. The data processing system 1301 provides a visual display 1305 for displaying the received image data.

The camera 501 may be a video camera and may continually supply video images to the data processing system, which can be viewed on the visual display 1305. After positioning the apparatus, as described with reference to FIG. 1 and FIG. 2, and upon activation of the manual activation button 201, a high definition still image may be viewed on the visual display 1305. In addition, a display may also be provided to show a graphical representation of the received electric field data. Furthermore, in an embodiment, it is possible for a visual image derived from the image data to be enhanced in response to the electric field data. This enhancement may be used to provide a local indication of the extent to which the skin condition may be considered malignant and therefore in need of further investigation. The electric field data and the image data may also be conveyed to the remote data analysis system 1302.

In an embodiment, a machine learning exercise may be performed to analyze image data and electric field data in combination with independent assessments, such that it is possible to then provide an additional indication of a need for medical intervention. Furthermore, over a period of time, as more data is collected, enhancements may be made to the machine learning process and the output from the machine learning process may be combined with local assessments made by operatives. This combined data may then allow enhancements to be made to the procedures performed by the data processing system 1305 and the remote data analysis system 1302.

In an alternative embodiment, data collected by the examining apparatus 101 may be conveyed, possibly via a data processing system, directly to the remote data analysis system. This allows sophisticated analysis to take place, possibly derived from a machine learning process, and in turn an indication may be returned from the remote data analysis system to the examining apparatus to indicate whether the skin condition under consideration may be considered as benign or malignant.

In in an embodiment, appropriately colored light emitting diodes may be included on the examination apparatus, resulting in the examining apparatus, for example, being illuminated in a blue color if the area under consideration is considered to be benign and illuminated in a red color if the area under consideration is considered to be malignant and thereby requiring further attention. Further colors may be included to indicate gradations between these two extremes. Alternatively, it is possible for a display device to be included as part of the examining apparatus itself.

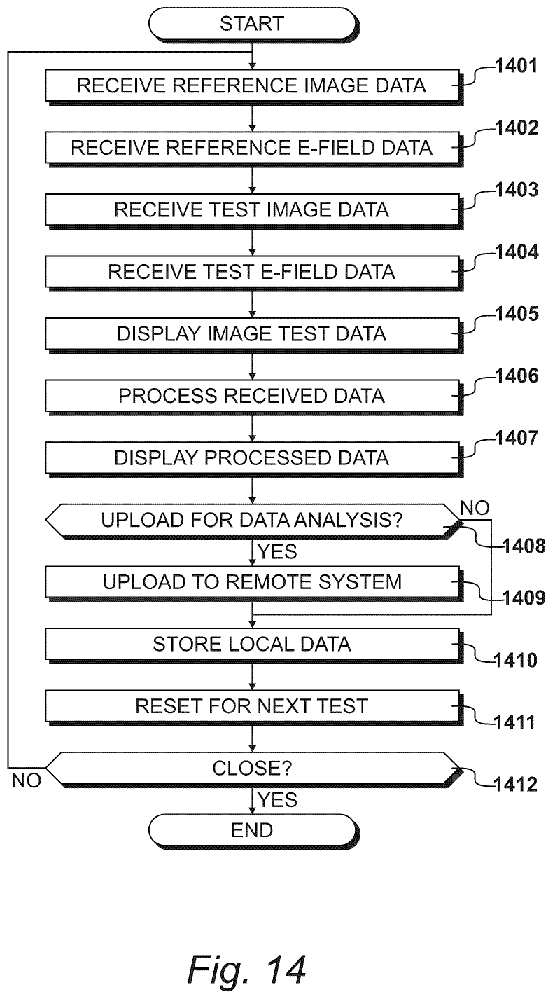

### FIG. 14

Procedures performed by the data processing system 1301 are illustrated in FIG. 14. At step 1401, reference image data is received whereafter, at step 1402, reference electric field data is received. The selected images are stored in the examination apparatus 101 and the electric field data is read from the database table described with reference to FIG. 12. Thereafter, at step 1403, test image data is received and at step 1404 test electric field data is received.

At step 1405 the image test data is displayed on the visual display 1305. The test image data is captured in response to operation of the manual activation button 201 as described with reference to FIG. 2.

At step 1406 the data that has been received at steps 1401 to 1404 are processed and the processed data is displayed at step 1407.

At step 1408 a question is asked as to whether the data is to be uploaded for data analysis and when answered in the affirmative, the data is uploaded to the remote data analysis system 1302. This allows an operative to perform the exercise several times upon the same subject until data has been collected that is considered optimum by the operative. Thus, a graphical user interface displayed on the visual display 1305 may invite an operative to upload the data for data analysis or invite the operative to repeat the examination process.

At step 1410 the local data that has been uploaded to the remote system, at step 1409, is stored locally on the data processing system 1301 whereafter, at step 1411, the system is reset for the next testing operation to be performed. At step 1412 a question is asked as to whether the process is to close and the process ends if the question is answered in the affirmative. Alternatively, control returns to step 1401 in anticipation of receiving further data.

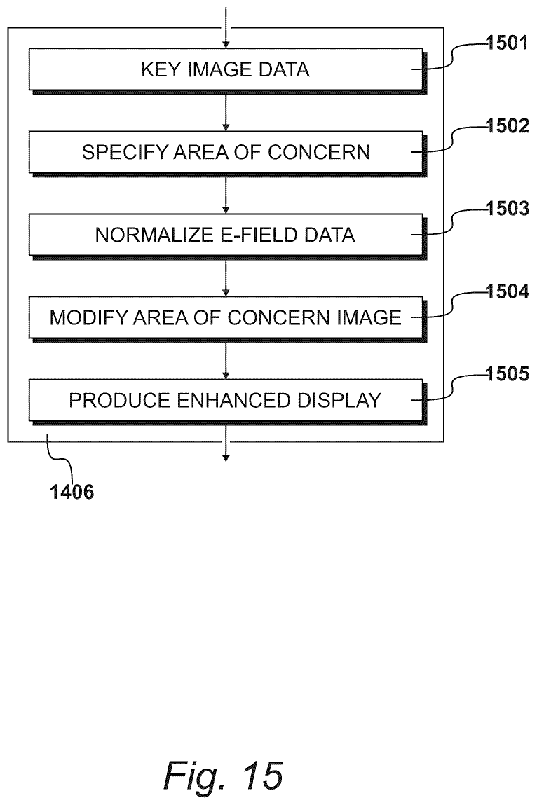

### FIG. 15

An example of the procedures 1406 for processing the received data are shown in FIG. 15. However, it should be appreciated that many manipulations of this data may be performed to facilitate local assessments and to optimize the quality of the data that is uploaded to the remote data analysis system.

In this example, the image data is keyed at step 1501 to separate the image of the visible skin condition from background skin. This allows the area of concern to be specified at step 1502.

At step 1503 the electric field data is normalised, which may involve subtracting the reference data from the test data. Again, alternative mathematical manipulations may be performed at this stage with a view to optimizing the effectiveness of the electric field data.

Fundamentally, the purpose of these procedures is to identify the existence of problematic conditions. Thus, the electric field data may suggest that the area of skin which shows a visible skin condition does not represent an area of skin that may be considered problematic, which may in turn suggest that no further action is required. Alternatively, the electric field test data may be substantially different from the electric field reference data and this in turn may indicate that the skin condition is malignant and requires further attention. Consequently, in this embodiment, the area of concern in the image data is modified at step 1504 and an enhanced display of this modified data is produced at step 1505.

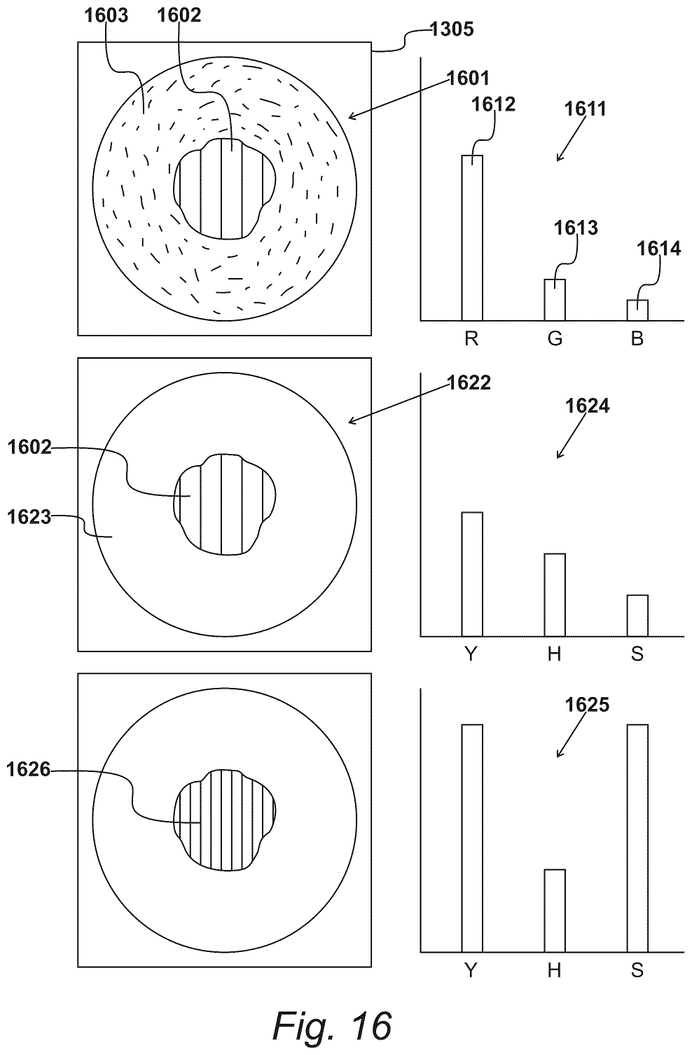

### FIG. 16

The results of the procedures described with reference to FIG. 15 are illustrated in FIG. 16. Images are displayed on the visual display 1305, either sequentially, as shown in FIG. 16, or in combination.

A first image 1601 represents the image test data displayed at step 1405. This shows the particular skin condition of interest 1602 surrounded by healthy skin 1603. The raw image data consists of eight or ten bits representing light intensities for red, green and blue. These are illustrated in a first histogram 1611, depicting a level for red 1612, a level for green 1613 and a level for blue 1614. These levels represent the color of the skin condition 1602 and different levels would be represented for the surrounding skin 1603. These differences allow the area representing the skin condition 1602 to be distinguished over values representing the background skin 1603. Following known keying techniques, this allows pixels representing the skin condition to be separated from the background pixels, such that the image may depict the skin condition against a plain white background as shown in a second image 1622. In the second image 1622, the skin condition image 1602 appears as before but this time it is shown against a white background 1623.

When represented in red-green-blue (RGB) color space, pixel values representing the skin condition 1602 remain the same as illustrated in chart 1611. As is known in the art, manipulations may be performed within this RGB color space.

In this embodiment, the intention is to enhance pixel values which show the skin condition to an extent determined by the analysis of the electric field data. In this example, individual pixel values are transformed from red-green-blue color space to luminance, hue and saturation color space (usually identified as YHS respectively). A second histogram 1624 shows the same color as that represented in chart 1611 but in YHS color space. Furthermore, in this embodiment, modifications are made at step 1504 by adjusting the luminance values and the saturation values in response to the electric field data.

For the purposes of this example, it is assumed that the electric field test data differs significantly from the electric field reference data. This creates a significant difference signal and this is used to make an appropriate adjustment. Thus, as shown in a third histogram 1625, the luminance of the image has been significantly increased and the saturation of the image has also been significantly increased. The hue remains unchanged such that the color of the displayed region 1626 substantially retains its original color.

Referring to the first histogram 1611, the color is substantially red and therefore appears red both in the original image and in the processed image. However, based upon the electric field difference values, the brightness of the image 1626 is increased and the saturation of the image is also increased. Adjustments to the saturation will make the red color appear more red and less pink for example. An operative can clearly compare the modified image with the original image and from this make an assessment as to whether further intervention is required. Furthermore, additional prompts may be provided by the data processing system itself and a recommendation may also be made to the extent that the data should be sent to the remote data analysis system.

In alternative embodiments, different modifications to the image data could be made. For example, the hue of the image could be changed making the area of the visible skin condition appear in a very unsightly green color, for example, when a malignant condition is suspected. Alternatively, the luminance value could be modified periodically to create a flashing or throbbing image; again, bringing the condition to the attention of an operative.

The system therefore presents a method of examining a visible skin condition in which a housing is located at a region of a visible skin condition. Image data is captured of the skin over the region and electric fields are generated that penetrate the skin over the region, such that an application of the apparatus produces both image related signals and electric field related signals for the same skin condition.

In an embodiment, a visual display is created that is derived from the image data and this image may then be enhanced in response to the electric field data. In an embodiment, the test image data is compared against reference image data to identify an area of concern within the region, as shown in image 1622. Furthermore, a keying operation may be performed to isolate the area of concern in the image data and colour properties of the area of concern may be changed in response to the electric field data.

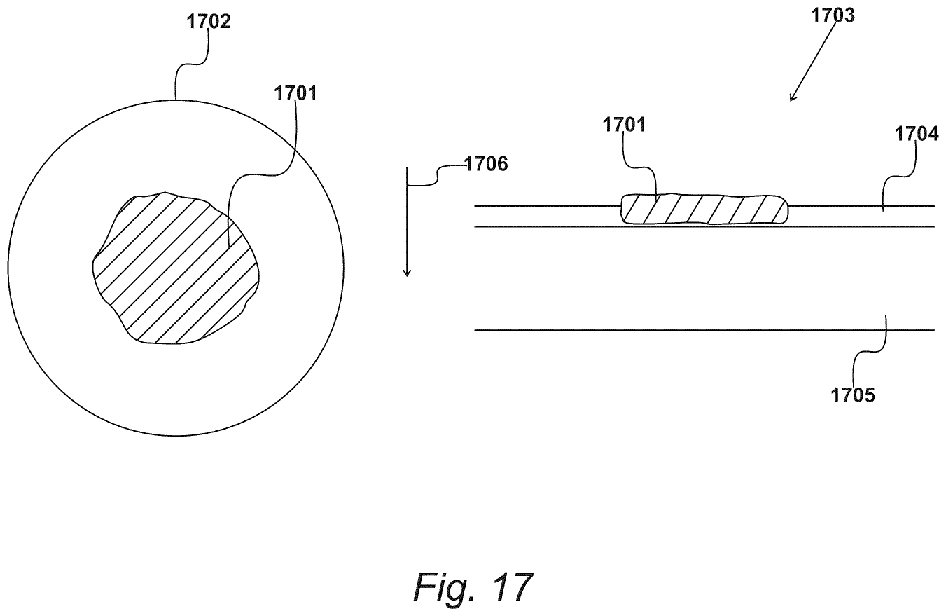

### FIG. 17

An image of a visible skin condition 1701 is shown in FIG. 17. The apparatus described with reference to FIG. 2 has been located over this visible skin condition and a captured image is bounded by circle a 1702. For each deployment of the apparatus, the size of the visible image remains substantially constant and the number of pixels populated also remains constant. Consequently, the overall area of the image contained within the circle 1702 is known and may be represented in terms of the number of pixels present.

Following a keying operation, as described with reference to FIG. 16, it is possible for the area of the visible skin condition 1701 to be determined, as a subset of the available pixels, such that the area may be converted into conventional units or represented as a percentage of the overall area contained within circle 1702. For the purposes of this illustration, it may be assumed that the visible skin condition 1701 is relatively large and, to a clinician, could be seen as problematic. The visible skin condition may be identified as a mole for example and the clinician would wish to know the extent to which the mole has penetrated the skin, representing the possibility of the undesirable material reaching a blood supply.

For the purposes of this illustration, a cross-section of the visible skin condition 1701 is shown at 1703. The cross-section illustrates an epidermis 1704 and a dermis 1705. In many situations, moles of this type would be considered problematic if they have, or if they possibly could, penetrate the dermis 1705. However, in this example, the depth of the visible skin condition 1701, as illustrated by a first arrow 1706, is relatively shallow and has not entered the dermis 1705. However, the captured image data does not provide this information and a clinician may be prompted to organise a biopsy. However, in the environment of FIG. 17, this would be unnecessary and the clinician would be better informed if it were possible to obtain information indicating the depth of penetration without incurring a surgical procedure.

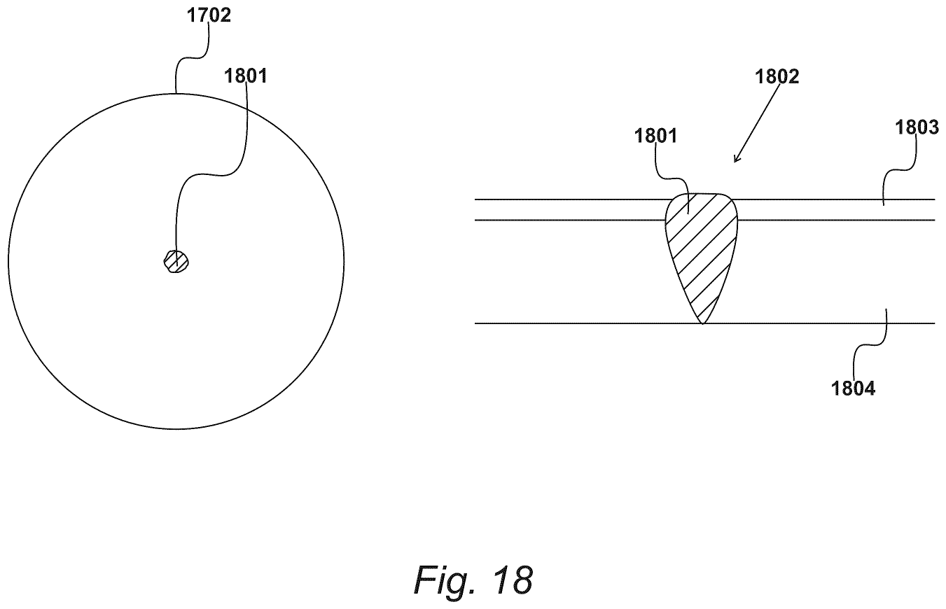

### FIG. 18

An image of a second visible skin condition 1801 is shown in FIG. 18. The overall area of the totality of the image is substantially the same as that described with reference to FIG. 17 and is again identified by circle 1702. Again, the area of the second visible skin condition 1801, which again may be identified as a mole, is much smaller than the area of the first visible skin condition 1701. Thus, when presented only with the image data, a clinician may be tempted to conclude that the first visible skin condition 1701 appears more problematic than the second skin condition 1801.

For the purposes of illustration, a cross-section of the second skin condition is shown at 1802, with similar representations for an epidermis 1803 and a dermis 1804. In this example, the second visible skin condition 1801 has penetrated through the epidermis 1803 and has also penetrated through most of the dermis 1804; such that there is now a significant risk of the undesirable material reaching blood capillaries below the dermis 1804. Thus, although from a visual inspection, the first visible skin condition 1701 appears more problematic than the second visible skin condition 1801, in reality, due to the depth of penetration, the second visible skin condition 1801 is actually more problematic and would be assessed, following biopsy, as requiring surgical intervention.

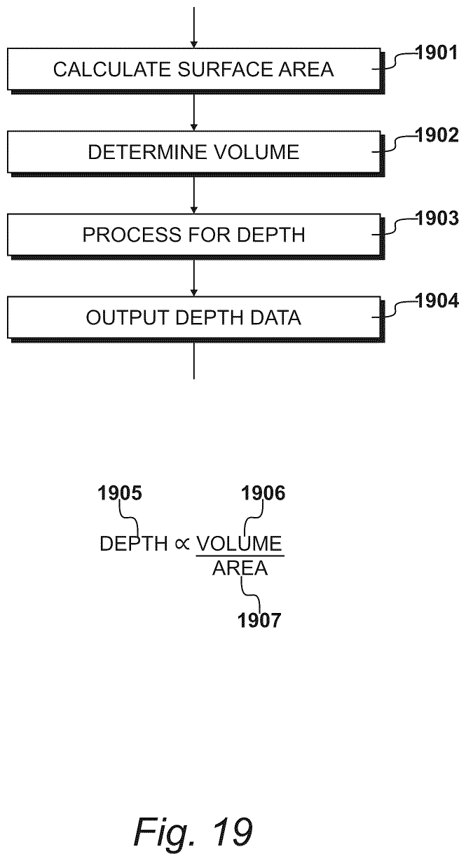

### FIG. 19

A further embodiment for step 1406 of processing the received data, as described with reference to FIG. 14, is illustrated in FIG. 19. In an embodiment, steps described with reference to FIG. 15 may also be incorporated.

At step 1901 the surface area of the visible skin condition is calculated. This makes use of the captured image data as described with reference to FIG. 17.

At step 1902, the volume of the visible skin condition is determined from the electric field related data. Thereafter, at step 1903, the calculated surface area is processed in combination with the determined indication of volume to assess the depth of skin penetration.

As illustrated in FIG. 3, the generated electric fields extend from the plane of the apparatus in three-dimensional space and thereby penetrate the skin. In an embodiment, this degree of penetration is such that it extends below the dermis 1705/1804. As previously described, data is obtained by generating reference data which is then compared with the test data. The inventor has appreciated that the resulting electric field related signals represent, in the test data, values which vary with respect to the amount or volume of undesirable material present. Thus, the electric field data does not exclusively vary with respect to the viewable area of the skin condition but varies with respect to the totality of material present in the three-dimensional region comprising the epidermis 1803 and the dermis 1804.

For the purposes of this illustration, it may be assumed that the volume of the undesirable material identified at 1701 is substantially similar to the volume of undesirable material identified at 1801. In many practical implementations, it is unlikely that these assessments will be perfectly linear but in alternative embodiments, modifications may be made to the geometry to improve linearity, processing exercises may be performed to compensate for non-linear effects or reliance may be made upon a trained machine learning procedure. However, the realisation made by the inventor is to the effect that an indication of depth 1905 may be obtained by dividing the determined volume 1906 by the calculated surface area 1907.

Following this procedure, the first visible skin condition 1703 produces a determined volume that is very similar to that of the second visible skin condition 1802. However, the calculated area for the first visible skin condition 1701 is much larger than the calculated area for the second visible skin condition 1801, therefore the assessment of depth 1905 will be significantly deeper for the second visible skin condition 1801. Consequently, in a clinical environment, an operative would receive information to the effect that the second visible skin condition 1801 is actually likely to be more problematic than the first visible skin condition 1701.

In an embodiment, deploying procedures substantially similar to those described with reference to FIG. 15 and FIG. 16, modifications to the perceived color of the visible skin conditions could be changed with reference to the depth assessment 1905. Thus, for the purposes of illustration, it may be assumed that when viewing the captured image data, the first visible skin condition 1701 appears to have a color that is substantially similar to the second visible skin condition 1801. However, following the procedure described with reference to FIG. 19, color modifications may be made, as described with reference to FIG. 16, such that, when viewed, greater modifications are made to the color of the second visible skin condition 1801 compared to the first visible skin condition 1701.

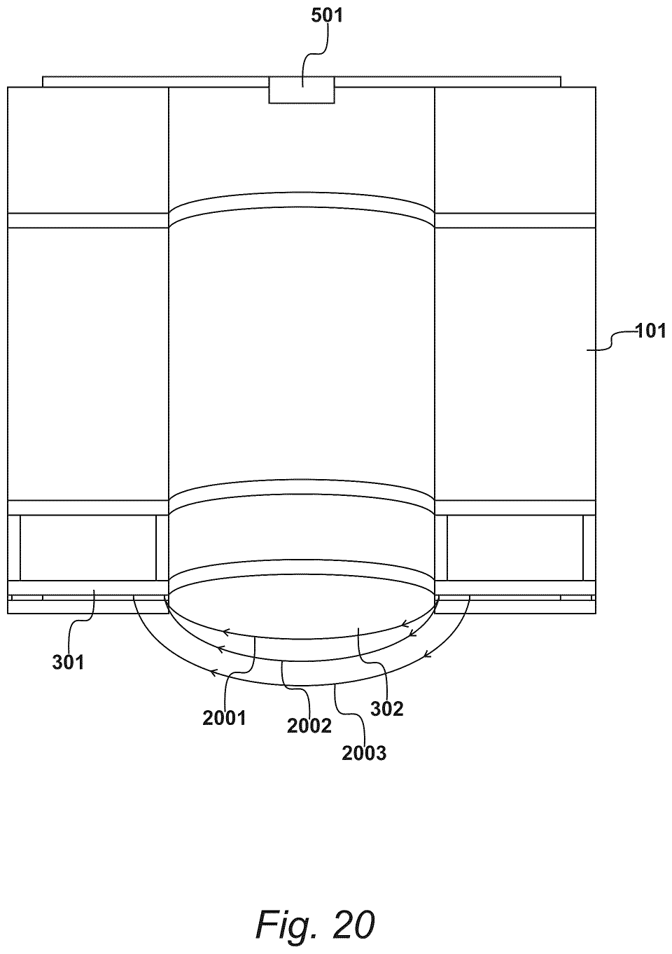

### FIG. 20

A cross-section of the apparatus of FIG. 3 is shown in FIG. 20. The housing 101 supports the first dielectric substrate 301 and the camera 501. The visible skin condition is viewed through the hole 302 in the first dielectric substrate 301. Scanning electrodes on the first dielectric substrate sequentially produce electric fields including a first electric field 2001. This extends vertically from the housing 101 to penetrate the visible skin condition. Subsequently, a second electric field 2002 is generated that also penetrates the visible skin condition but with a greater degree of penetration. Similarly, a third electric field 2003 is generated with an even greater degree of skin penetration.

The invention claimed is:

1. A method of examining a visible skin condition, comprising the steps of:
locating a housing at a region of said visible skin condition;
capturing image data of skin over said region;
generating electric fields that penetrate said skin over said region, such that an application of an apparatus produces both image-related signals and electric field related signals for a same skin condition;
assessing depth of skin penetration of said visible skin condition; and
generating an output indicative of said assessing step, comprising the steps of:
calculating a surface area of said visible skin condition from said image data produced by said capturing step;
determining an indication of a volume of said visible skin condition from said electric field related signals; and
processing said calculated surface area in combination with said determined indication of said volume to assess said depth of said skin penetration.

2. The method of claim 1, wherein:
said generating step is performed by an electric field generating device that comprises a first dielectric substrate having a hole having a first radius;
said capturing step is performed by a camera; and
said visible skin condition is viewed through said hole.

3. The method of claim 2, wherein said image data is captured by said camera substantially at a same time as said electric fields are generated by said electric field generating device.

4. The method of claim 1, wherein said calculating step is performed by pixel counting.

5. The method of claim 1, further comprising the step of locating said housing over an area of healthy skin to obtain reference signals for comparison against test signals.

6. The method of claim 1, wherein said image data from said capturing step is displayed on a visual display device.

7. The method of claim 6, further comprising the step of enhancing said image data in response to electric field data yielded from said electric field related signals.

8. The method of claim 7, comprising the step of changing color properties of said visible skin condition shown in said image data in response to said electric field data.

9. The method of claim 7, comprising the step of changing said image data by an extent related to said assessed depth of said skin penetration.

10. The method of claim 1, further comprising the step of analysing said image data and electric field data yielded from said electric field related signals to produce an indication of a need for medical intervention with respect to said visible skin condition, following a machine learning exercise.

11. An apparatus for examining visible skin conditions, comprising a housing locatable at a region of a skin condition, wherein said housing contains:
a camera for capturing image data of skin over said region; and
an electric field generating device configured to generate electric fields that penetrate said skin over said region, such that an application of the apparatus produces both image related signals and electric field related signals for a same skin condition, wherein:
said electric field generating device comprises a first dielectric substrate with a hole having a first radius; and
said camera views said skin condition through said hole.

12. The apparatus of claim 11, wherein said camera is configured to capture said image data substantially at a same time as said electric field generating device generates said electric fields.

13. The apparatus of claim 11, comprising a processor configured to:
assess a depth of skin penetration of said skin condition; and
generate an output indicative of said assessing step.

14. The apparatus of claim 13, wherein said processor assesses said depth of said skin penetration and is configured to:
calculate a surface area of said skin condition from said image data;
determine an indication of a volume of said skin condition from said electric field related signals; and
process said calculated surface area in combination with said determined indication of said volume to assess said depth of said skin penetration.

15. The apparatus of claim 11, wherein respective electrical conductors from each scanning electrode of said electric field generating device are configured to pass through said first dielectric substrate to a second planar surface.

16. The apparatus of claim 11, comprising a plurality of circumferentially and substantially evenly displaced secondary electrodes.

17. The apparatus of claim 11, further comprising a signal processor for processing said electric field related signals produced by said electric field generating device, influenced by electrical properties of penetrated tissue, to produce electric field data.

18. The apparatus of claim 11, comprising a visual display device for showing said image data.

19. The apparatus of claim 18, wherein a visual image derived from said image data is enhanced in response to electric field data yielded from said electric field related signals.

20. The apparatus of claim 11, further comprising an analysing processor configured, following a machine learning exercise, to analyse said image data and electric field data yielded from said electric field related signals to produce an indication of a need for medical intervention with respect to said skin condition.
